(19) United States
(12) Patent Application Publication
(10) Pub. No.: US 2007/0071733 A1
(43) Pub. Date: Mar. 29, 2007

(54) Synthetic substrate for tissue formation

(75) Inventors: Rita Kandel, Toronto (CA); Robert Pilliar, Toronto (CA)

Correspondence Address: HOWSON AND HOWSON, SUITE 210, 501 OFFICE CENTER DRIVE, FT WASHINGTON, PA, 19034, US

(73) Assignee: Mount Sinai Hospital, Toronto (CA)

(21) Appl. No.: 11/581,606

(22) Filed: Oct. 16, 2006

Related U.S. Application Data
(63) Continuation of application No. 10/110,547, filed on Sep. 16, 2002, now abandoned.
(60) Provisional application No. 60/159,845, filed on Oct. 15, 1999.

(51) Int. Cl. A61K 35/32; C12N 5/08

(52) U.S. Cl. 424/93.7; 424/423; 435/325; 435/366

(57) ABSTRACT

The invention relates to a substrate on which to grow synthetic cartilage, a method for preparing the substrate, a synthetic cartilage patch comprising the substrate, and methods of using the synthetic cartilage patch.

### FIELD OF THE INVENTION

The invention relates to a substrate on which to grow synthetic cartilage, a method for preparing the substrate, a synthetic cartilage patch comprising the substrate, and methods of using the synthetic cartilage patch.

### BACKGROUND OF THE INVENTION

A number of different approaches have been developed to treat mammalian articular cartilage defects. Tissue engineering approaches have been investigated for the resurfacing of localized damaged regions of joints. One approach uses porous synthetic material forms as substrates for cartilage formation in vitro. Studies have suggested that substrate material characteristics can influence chondrocyte phenotype and the extracellular matrix formed (Grande D et al., J Biomed Matl Res, 34:211-220, 1997 and Nehrer S. et al., Biomaterials, 18: 769-776, 1997). Defining optimal substrate material characteristics will provide improved substrates for use in the repair of articular cartilage defects in mammals.

### SUMMARY OF THE INVENTION

The invention provides a substrate on which to row synthetic cartilage comprising a porous construct with interconnected pores having an average pore size less than 70 μm, preferably less than 40 μm, more preferably less than 20 μm, most preferably less than 15 μm, to permit growth of the synthetic cartilage. The invention also provides a method for producing a substrate on which to form or grow synthetic cartilage comprising producing from a material capable of forming pores with a selected pore size a porous construct with interconnected pores having an average pore size less than 70 μm, preferably less than 40 μm, more preferably less than 20 μm, most preferably less than 15 μm. In an embodiment, the average pore size is between 10 and 40 μm, preferably between 10 and 30 μm, more preferably between 10 and 20 μm, and most preferably between 10 and 15 μm.

The invention also contemplates a substrate comprising (a) a surface component on which to grow synthetic cartilage comprising a porous construct with interconnected pores having an average pore size less than 70 μm, preferably less than 40 μm, more preferably less than 20 μm, most preferably less than 15 μm, to permit growth of synthetic cartilage thereon, and (b) a deeper component comprising a porous construct with a pore size selected to permit bone ingrowth into the substrate. The deeper component facilitates or favors bone ingrowth into the substrate after implantation.

The invention also relates to a synthetic cartilage patch for the repair of a cartilage defect in a mammal in vivo comprising synthetic cartilage formed on, or in combination with, a substrate of the invention. The substrate enables a greater amount of tissue formation.

The invention also contemplates a method for preparing in vitro a synthetic cartilage patch, preferably a synthetic articular cartilage patch, for the repair of a cartilage defect in a mammal. The method comprises (a) producing from a material capable of forming pores with a selected pore size, a porous construct with interconnected pores having an average pore size less than 70 μm, preferably less than 40 μm, more preferably less than 20 μm, most preferably less than 15 μm; and (b) culturing denuded chondrogenic cells on the substrate under conditions sufficient to permit the cells to form a three-dimensional multi cell-layered patch of synthetic cartilage.

In an aspect the invention provides a method for effecting the repair of a cartilage defect at a pre-determined site in a mammal comprising (a) surgically implanting at the pre-determined site a synthetic cartilage patch of the invention; and (b) permitting the synthetic cartilage of the patch to integrate into the pre-determined site.

Further, the invention provides a system for testing a substance that affects cartilage tissue comprising: culturing denuded chondrogenic cells on a substrate of the invention under conditions to permit the cells to form a three-dimensional multi cell-layered patch of synthetic cartilage in the presence of a substance which is suspected of affecting formation or maintenance of cartilage, and determining the biochemical composition and/or physiological organization of the synthetic cartilage generated in the culture with the biochemical composition and/or physiological organization of the synthetic cartilage in the absence of the substance.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

The invention will be better understood with reference to the drawings in which:

FIG. 1 shows photomicrographs of cartilagenous tissue formed on Ti6Al4V discs of average pore size A) 13 μm, B) 43 μm, C) 68 μm (toluidine blue, magnification×100);

FIG. 2 is a bar graph showing DNA content of a cartilagenous tissue formed on titanium alloy (Ti6Al4V) of different average pore size from a representative experiment; and

FIG. 3 is a bar graph showing proteoglycan content of a cartilagenous tissue formed on titanium alloy (Ti6Al4V) of different average pore size from a representative experiment.

### DETAILED DESCRIPTION OF THE INVENTION

The invention provides a substrate on which to grow synthetic cartilage comprising a porous construct with interconnected pores having an average pore size less than 70 μm, preferably less than 40 μm, more preferably less than 20 μm, most preferably less than 15 μm, to permit growth of the synthetic cartilage. The invention also provides a method for producing a substrate on which to form or grow synthetic cartilage comprising producing from a material capable of forming pores with a selected pore size, a porous construct with interconnected pores having an average pore size less than 70 μm, preferably less than 40 μm, more preferably less than 20 μm, most preferably less than 15 μm. In an embodiment of the invention, the material is a powder. In a preferred embodiment, the powder is sintered under suitable conditions to fuse particles of the powder (i.e. powder particles) to form a porous construct with the properties of a substrate of the invention.

A substrate of the invention may also comprise a deeper component for mated engagement with a mammalian bone. The pore size of the deeper component is selected to facilitate or favor bone ingrowth into the substrate after implantation into a mammal. Thus, the substrate may comprise (a) a surface component

comprising a porous construct with interconnected pores having an average pore size less than 70 μm, preferably less than 40 μm, more preferably less than 20 μm, most preferably less than 15 μm, to permit growth of the synthetic cartilage thereon, and (b) a deeper component comprising a porous construct with a larger average pore size compared to (a) selected to permit bone ingrowth into the substrate. In an embodiment, the pore size of the deeper component is between about 30 to 200 μm.

A substrate of the invention may be used for forming other soft tissues including but not limited to connective tissue, intervertebral disc, fibrous tissue, tendons, and ligaments.

The invention also relates to a synthetic cartilage patch for the repair of a cartilage defect in a mammal in vivo comprising synthetic cartilage formed on, or in combination with, a substrate of the invention. The substrate enables a greater amount of tissue formation. In particular, the synthetic cartilage is characterized by higher cellularity (about two fold higher, in particular on average 1.5 fold higher) and higher proteoglycan content (about two fold higher, in particular, on average 1.5 fold higher) as compared to the tissue formed on substrates with interconnected pores having an average pore size of about 40 μm or greater.

The invention also contemplates a method for preparing in vitro a synthetic cartilage patch, preferably a synthetic articular cartilage patch, for the repair of a cartilage defect in a mammal. The method comprises (a) preparing a substrate comprising forming from material capable of forming pores with a selected pore size, a porous construct with interconnected pores having an average pore size less than 70 μm, preferably less than 40 μm, more preferably less than 20 μm, most preferably less than 15 μm; and (b) culturing denuded chondrogenic cells on the substrate under conditions sufficient to permit the cells to form a three-dimensional multi cell-layered patch of synthetic cartilage. The resulting synthetic cartilage contains chondrogenic cells dispersed within a matrix. The synthetic cartilage is also characterized as having a higher cellularity as demonstrated by higher DNA content, and a higher proteoglycan content when compared to synthetic cartilage formed on substrates having a greater average pore size (i.e. greater than about 40 μm) or formed from powders with a higher powder size (greater than about 45 μm). In step (a), the porous construct may be formed with or on a deeper component as described herein, or it may be placed on a preformed deeper component.

The substrate may be a preformed structure containing a surface component and optionally a deeper component, or it may be a composite construction of the two components. The surface component and deeper component may be formed as separate stages or as an integral structure.

The material (e.g. powder) used to prepare a substrate of the invention may be based on pure titanium or titanium alloy (e.g. Ti6Al4V), hydroxyapatite, calcium carbonate, calcium phosphate (see PCT/CA97/00331 published as WO97/45147, and U.S. Pat. No. 6,077,989), or other like inorganic materials. The particle size of a powder used to prepare a surface component porous construct is selected to provide a pore size of less than 70 μm, preferably less than 40 μm, more preferably less than about 20 μm, most preferably less than 15 μm. A suitable particle size is less than 100 μm, more preferably less than 50 μm, most preferably less than 45 μm.

In a method of the invention a powder (e.g. powders of calcium phosphates, titanium or titanium alloy (Ti6Al4V), hydroxyapatite, or calcium carbonate) is used to form the substrate. The powder can be sintered, as for example, by pressure or gravity sintering just below the melting temperature of the material, or at a temperature below the melting temperature of the material but above a temperature to allow sufficient atom or molecule diffusion or viscous flow to allow the formation of significant neck regions between particles. This will produce a surface component porous construct having interconnected pores with average pore sizes of less than 70 μm, preferably less than 40 μm, more preferably less than 20 μm, most preferably less than 15 μm. The particle size of the powder is selected to provide the desired pore size which for the surface component is typically less than 100 μm, more preferably less than 50 μm, most preferably less than 45 μm.

It will be appreciated that other methods and materials known in the art can be used to prepare substrates with selected pore sizes (e.g. laser sintering, direct solidification, sintering of fibers, bonding meshes, and preferential dissolution of sacrificial elements). See for example, PCT/CA97/00331 (published as WO97/45147), and U.S. Pat. No. 6,077,989.

A substrate of the invention may be formed into any size or shape, preferably one suitable for forming a synthetic cartilage patch for implantation in a mammal. For example, a substrate may be formed into rods, pins, discs, screws, and plates, preferably discs, that may be cylindrical, tapered, or threaded. The resulting patch may interfit directly into a cartilage defect, or it may be trimmed to the appropriate size and shape prior to insertion into the defect.

The term “synthetic cartilage” used herein refers to any cartilage tissue produced in vitro that contains chondrogenic cells dispersed within an endogenously produced and secreted extracellular matrix, including but not limited to synthetic articular cartilage. The extracellular matrix is composed of collagen fibrils, sulfated proteoglycans e.g. aggrecan, and water.

“Synthetic articular cartilage” refers to any cartilage tissue produced in vitro that biochemically and morphologically resembles the cartilage normally found on the articulating surfaces of mammalian joints.

The term “chondrogenic cells” refers to any cell which when exposed to an appropriate stimuli can differentiate into a cell capable of producing and secreting components characteristic of cartilage tissue, for example, fibrils of type II collagen, and large sulfated proteoglycans. Chondrogenic cells used in the practice of the invention may be isolated from any tissue containing chondrogenic cells. The chondrogenic cells can be isolated directly from pre-existing cartilage tissue, including hyaline cartilage, elastic cartilage, or fibrocartilage. In particular, the chondrogenic cells can be isolated from articular cartilage (from either weight bearing or non-weight bearing joints), costal cartilage, sternal cartilage, epiglottic cartilage, thyroid cartilage, nasal cartilage, auricular cartilage, tracheal cartilage, arytenoid cartilage, and cricoid cartilage. Chondrogenic cells, specifically mesenchymal stem cells, can also be isolated from bone marrow using techniques well known in the art (see for example, Wakitani et al, 1994, J. Bone Joint Surg. 76: 579-591, U.S. Pat. Nos. 5,197,985 and 4,642,120).

Preferably the chondrogenic cells are isolated from articular cartilage. Biopsy samples of articular cartilage can be isolated during arthroscopic or open joint surgery using procedures well known in the art (See Operative Arthroscopy 1991, McGinty et al., Raven Press, New York).

The chondrogenic cells may be isolated from mammals, preferably humans, bovines, ovines, rabbits, equines, most preferably humans. The chondrogenic cells can be isolated from adult or fetal tissue. In an embodiment of the invention, the chondrogenic cells are isolated from the metacarpal-carpal joints of calves as described in Boyle J. et al. (Osteoarthritis and Cartilage, 3: 117-125, 1995).

The chondrogenic cells may be transformed with recombinant vectors containing an exogenous gene encoding a biologically active protein which corrects or compensates for a genetic deficiency.

A “denuded cell” refers to any cell that has been isolated from a disaggregated tissue containing such a cell. A tissue can be enzymatically and/or mechanically disaggregated in order to release denuded cells. Conventional methods can be used to isolate chondrogenic cells from tissues. For example, the chondrocytes may be isolated by sequential enzyme digestion techniques using proteolytic enzymes including chondroitinase ABC, hyaluronidase, pronase, collagenase, or trypsin. In an embodiment, the present invention uses the method described in Kandel et al, Biochem. Biophys. Acta. 1035:130, 1990 or Boyle et al, J supra.

Chondrogenic cells are seeded (e.g. 1×10⁵ to 8×10⁸ cells/cm², more preferably 1×10⁶ to 8×10⁸ cells/cm², most preferably 1.5×10⁷ cells/cm²) on a substrate and grown under conventional culture conditions. For example, the cultures are grown in Hams F12 medium containing 5% fetal bovine serum, and after about seven days ascorbic acid (e.g. 100 μg/ml) is added to the medium. The cultures are then maintained (e.g. 1 to 100 days, preferably 1 to 60 days) to induce the production and accumulation of extracellular matrix and thus the formation of synthetic cartilage.

In an embodiment of the invention the chondrocytes are formed on a substrate using the methods described in U.S. Pat. No. 5,326,357 and PCT CA96/00729 (published as WO 97/17430).

A synthetic cartilage patch of the invention can be used as an implant to replace or repair cartilage defects. Defects can be readily identified during arthroscopic examination or during open surgery of the joint.

They can also be identified using computer aided tomography (CT scanning), X-ray examination, magnetic resonance imaging (MRI), analysis of synovial fluid or serum markers, or other procedures known in the art. Treatment of defects can be carried out during an arthroscopic or open joint procedure. Once a defect is identified it may be treated using a method of the invention.

The invention contemplates a method for effecting the repair of a cartilage defect, preferably an articular cartilage defect, at a pre-determined site in a mammal (preferably humans) comprising (a) surgically implanting at the pre-determined site a synthetic cartilage patch of the invention described herein; and (b) permitting the synthetic cartilage to integrate into the pre-determined site (e.g. into cartilage). The substrate portion of the synthetic cartilage patch may be fixed in place to bone, for example, using press fit, or an interlocking format (e.g. a threaded substrate). Where the substrate comprises a surface component and a deeper component, the deeper component is preferably implanted substantially in juxtaposition with bone. In some methods the defective cartilage is removed prior to implantation. A patch may be sized and shaped to fit the cartilage defect, or a plurality of patches can be implanted into the defect.

A synthetic cartilage patch may be assayed biochemically or morphologically using conventional methods well known to persons skilled in the art prior to implantation. For example, cell proliferation assays (Pollack, 1975, in “Readings in Mammalian Cell Culture”, Cold Spring Harbor Laboratory Press. Cold Spring Harbor), assays to measure chondrogenic potential of proliferated cells (e.g. agarose culture as described in Benya et al, 1982, Cell 30: 215-224), and biochemical assays and immunohistochemical staining may be used to confirm the composition of a synthetic cartilage patch.

A synthetic cartilage patch of the invention may be derived from allogeneic, xenogeneic, or preferably autogeneic cells. Synthetic allogeneic cartilage may be prepared from cells isolated from biopsy tissue, bone marrow aspirates, or serum samples from a mammal belonging to the same species as the recipient. Autogeneic patches can be prepared from cells obtained from biopsy sites from the intended recipient.

The methods described herein can be used in the treatment of both partial-thickness and full-thickness defects of articular cartilage. Full-thickness defects include changes in the articular cartilage, the underlying subchondral bone tissue, and the calcified layer of cartilage located between the articular cartilage and the subchondral bone. These defects can arise during trauma of the joint or during the late stages of degenerative joint diseases (e.g. osteoarthritis). Partial-thickness defects are restricted to the cartilage tissue itself and include fissures, clefts, or erosions. These defects are usually caused by trauma or mechanical derangements of the joint which in turn induce wearing of the cartilage tissue within the joint.

The invention still further relates to a system for testing a substance that affects cartilage tissue, preferably articular cartilage tissue, comprising: culturing denuded chondrogenic cells on a substrate of the invention under conditions to permit the cells to form a three-dimensional multi cell-layered patch of synthetic cartilage in the presence of a substance which is suspected of affecting formation or maintenance of cartilage, and determining the biochemical composition and/or physiological organization of the synthetic cartilage generated in the culture with the biochemical composition and/or physiological organization of the synthetic cartilage in the absence of the substance. The substance may be added to the culture, or the chondrogenic cells or synthetic cartilage may be genetically engineered to express the substance i.e. the chondrogenic cells may serve as an endogenous source of the substance.

The invention still further relates to a method of using the synthetic cartilage of the invention to test pharmaceutical preparations for efficacy in the treatment of diseases of the joint.

The invention also contemplates using the synthetic cartilage of the invention in gene therapy. Recombinant vectors containing an exogenous gene encoding a biologically active protein which is selected to modify the genotype and phenotype of a cell to be infected may be introduced into chondrogenic cells and accordingly in a synthetic cartilage patch of the invention. An exogenous gene coding for a biologically active protein which corrects or compensates for a genetic deficiency may be introduced into the cells and patch. For example, TIMP (tissue inhibitor of metalloproteases) can be introduced into the cells so that the cells secrete this protein and inhibit the metalloproteases synthesized by chondrocytes locally in diseases such as osteoarthritis and rheumatoid arthritis. A gene could also be inserted to metabolize iron which would be useful in the treatment of thalassaemia. The expression of the exogenous gene may be quantitated by measuring the expression levels of a selectable marker encoded by a selection gene contained in the recombinant vector.

Pharmaceutical agents and growth factors may be incorporated within the pores of a substrate of the invention. Thus, the invention contemplates the use of a synthetic cartilage patch of the invention to deliver pharmaceutical agents and growth factors.

The following non-limiting example illustrates the present invention:

### EXAMPLE

The present inventors discovered that cartilagenous tissue formed on substrates made from titanium alloy powders with particle sizes less than 100 μm, preferably less than 45 μm had greater cellularity and proteoglycan content as compared to tissue formed on discs made from intermediate powder size (45-150 μm) or from a larger powder size (>200 μm). Therefore, substrate structure as defined by pore size affects the amount of tissue formed as determined by the amount of proteoglycan accumulated.

Material and Methods

Materials: Porous Ti6Al4V discs of three different pore sizes were formed by sintering Ti6Al4V powders of three different size ranges; <45 um (average pore size ˜13 μm), 45-150 μm (average pore size ˜43 μm), and >200 um (average pore size ˜68 μm). Table 1 shows the average pore size and pore size distribution of the titanium discs. Each disc was 4.3 mm in surface diameter and 4 mm in height.

Chondrocyte Culture: Chondrocytes were isolated from full thickness articular cartilage obtained from the bovine metacarpal-carpal joint by sequential enzymatic digestion as described previously. The chondrocytes (2×10⁶ cells) were plated on the discs in Ham's F12 medium supplemented with 5% fetal bovine serum. On day 5 the fetal bovine serum concentration was increased to 20%, and on day 7 ascorbic acid (100 μg/ml, final concentration) was added. The cultures were maintained for 4 wks with medium changes every 2-3 days and fresh ascorbic acid added each time.

Histological Assessment of Chondrocyte Cultures: The cultures were harvested 4 weeks after plating, fixed in 10% buffered formalin and embedded in Osteobed®. Sections were cut and stained with toluidine blue.

Proteoglycan Content: Chondrocyte cultures were harvested at 4 wks and digested with papain [100 g/ml in 20 mM ammonium acetate, 11 mM EDTA, and 2 mM DTT] for at least 48 hrs at 65° C. The proteoglycan content was determined by measuring the amount of glycosaminoglycans in these digests using the dimethylmethylene blue dye binding assay and spectrophometry (Boyle, J. et al Osteoarthritis and Cartilage, 3:117-125, 1995).

DNA Content: Chondrocyte cultures were harvested at 4 wks and digested with papain as described above. The DNA content was measured using the Hoechst dye 33258 and fluorometry (Boyle, J. et al Osteoarthritis and Cartilage, 3:117-125, 1995).

Analysis of newly synthesized proteoglycans: Chondrocyte cultures were incubated with [³⁵S]SO₄ (8 μCi/disc) for 24 hrs prior to harvesting. Matrix proteoglycans were extracted with 4M guanidine HCl in 50 mM sodium acetate, pH 5.8 containing 0.1M 6-amino-hexanoic acid, 50 mM benzamidine HCl, 10 mM EDT . . . and 5 mM N-ethylmaleimide for 24 hrs at 4° C. Proteoglycan monomer size (Kav) was determined using Sepharose CL-2B chromatography under dissociative conditions. Kav was determined by Kav=(Vₑ−Vₒ)/(Vₜ−Vₒ), where Vₜ=total volume, Vₒ=void volume, and Vₑ=elution volume. Vₜ was determined with [³⁵S]SO₄ and the void volume was determined with dextran sulphate.

Statistical Evaluation: Results are presented as means and standard deviation (SD). Paired Student's t-test was used to determine significance between selected groups and significance was assigned at p<0.05.

Results

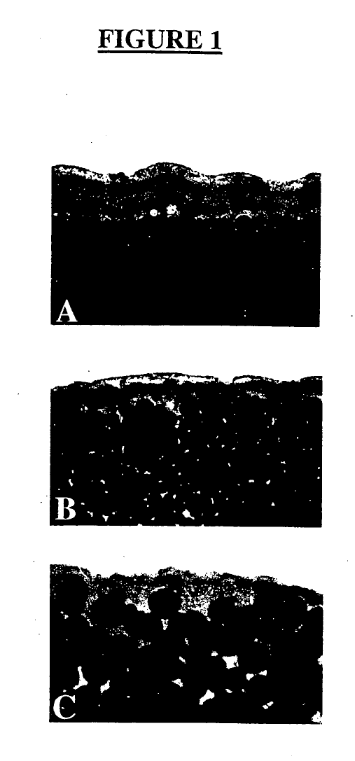

Examination of histological sections by light microscopy showed that cartilagenous tissue formed on the surfaces of the discs of all pore sizes within 4 weeks. The tissue formed on the Ti6Al4V discs made from powder size<45 μm appeared thicker than the tissue that formed on the discs made from powder sizes ranging between 45-150 μm or >200 μm (FIG. 1). Morphometric measurement of the tissue on the surface of the disc showed that the tissue formed on the Ti6Al4V disc of 13 μm average pore size was significantly thicker than the tissues that formed on the discs of larger average pore size (average thickness (μm)±SD: 13 μm average pore size=533±91; 43 μm average pore size=188±46; 68 μm average pore size=197±42). The pore size did not influence the size of proteoglycans synthesized by the chondrocytes (Kav±SD: powder size<45 μm=0.28±0.03; powder size 45-150 μm=0.29±0.03; powder size>200 μm=0.27±0.04). The Kav of these proteoglycans were similar in size to those synthesized by chondrocytes in ex vivo cartilage culture (Kav=0.27).

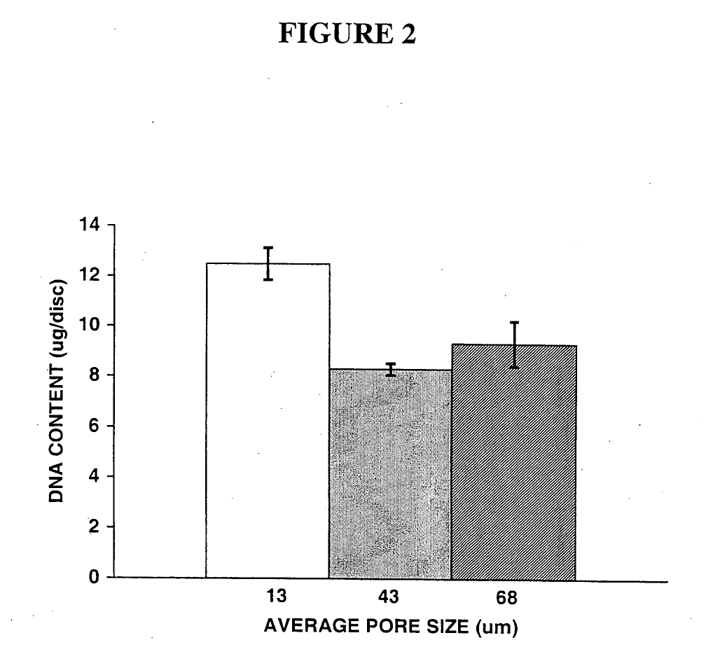

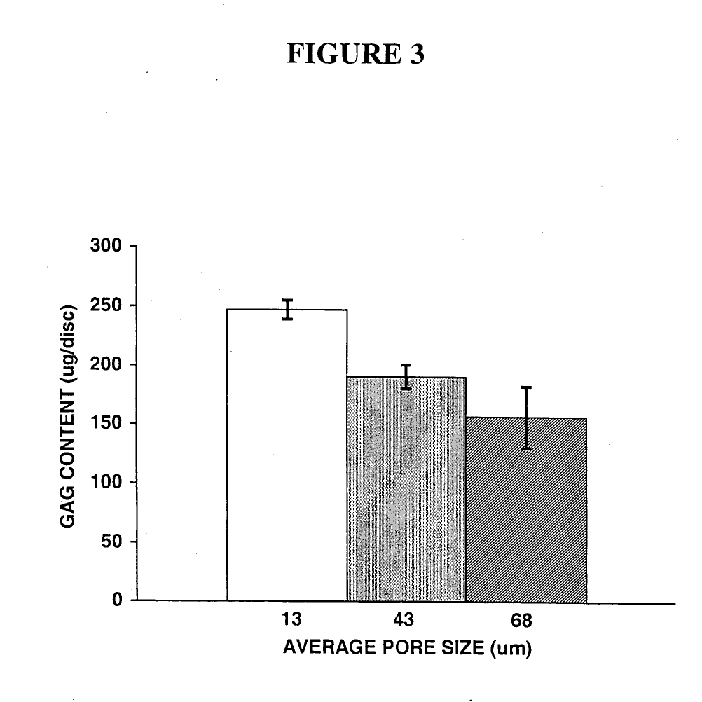

Biochemical analysis showed that the cartilagenous tissue formed on the Ti6Al4V discs made from the smallest powder size (<45 μm) was more cellular and had a DNA content of 12.5±0.6 μg/disc (FIG. 2). This was significantly greater than the DNA content of the cartilagenous tissue formed on the discs made from the intermediate powder sizes (45-150 μm) or the largest powder size (>200 μm) which showed similar amounts of DNA. In addition, cartilagenous tissue formed on the Ti6Al4V discs made from the smallest powder size (<45 μm) had a proteoglycan content of 246.9±8 μg/disc which was significantly greater than the proteoglycan content of the cartilagenous tissue formed on the discs of the intermediate powder size (45-150 μm) (190.4±10 μg/disc), or the largest powder size (>200 μm) (156.6±26 μg/disc) which had similar amounts of proteoglycan (FIG. 3). However, the amount of proteoglycan accumulated per cell was similar in the tissues formed on the discs made from the different powder sizes (GAG/DNA:<45 μm=18.7±0.4); 45-150 μm=19.8±4.2; >200 μm=20.5±4.8).

Pore size, within the range examined, had no effect on the size of proteoglycans synthesized nor the amount of proteoglycan accumulated per cell. However, the cartilagenous tissue that formed on the discs with an average pore size of 13 μm had greater cellularity and proteoglycan content compared to the tissue that formed on discs of larger average pore size. These results indicate that substrate structure as defined by pore size can affect the amount and the composition of the matrix that accumulates.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. All publications, patents and patent applications mentioned herein are incorporated herein by reference for the purpose of describing and disclosing the methodologies etc. which are reported therein which might be used in connection with the invention. Nothing herein is to be construed as an admission that the invention is not entitled to antedate such disclosure by virtue of prior invention.

It must be noted that as used herein and in the appended claims, the singular forms “a”, “an”, and “the” include plural reference unless the context clearly dictates otherwise. Thus, for example, reference to “a gene” includes a plurality of such genes.

TABLE 1
Average pore size and pore size distribution of titanium discs as
determined by Mercury Porosimetry

MERCURY
POROSIMETRY

| MATERIAL AND | Pore Size | Average |
|---|---|---|
| POWDER SIZE | Distribution | Pore Size |
| (μm) | (μm) | (μm) |
| TiAl <45 um | 8-29 | 13 |
| TiAl 45-150 um | 8-111 | 43 |
| TiAl >200 um | 15-105 | 68 |

The average pore size and pore size distribution as determined by the method of mercury porosimetry (TiAl = titanium alloy, Ti6Al4V))

We claim:

1. A substrate for forming synthetic cartilage comprising a porous construct with interconnected pores having an average pore size less than 70 μm to permit growth of synthetic cartilage.

2. The substrate according to claim 1, wherein said synthetic cartilage is characterized by having a higher cellularity and higher proteoglycan content when compared to synthetic cartilage formed on substrates having an average pore size greater than 40 μm.

3. The substrate according to claim 1 comprising a porous construct with interconnected pores having an average pore size less than 40 μm to permit growth of synthetic cartilage.

4. The substrate according to claim 1, wherein the average pore size is between 10 and 40 μm.

5. The substrate according to claim 1, wherein the porous construct is formed from a powder of calcium phosphates, titanium or titanium alloy (Ti6Al4V), hydroxyapatite, or calcium carbonate.

6. The substrate according to claim 1 further comprising:
(a) a surface component on which to grow synthetic cartilage comprising a porous construct with interconnected pores having an average pore size less than 70 μm; and
(b) a deeper component comprising a porous construct with a pore size selected to permit bone ingrowth in the substrate.

7. The substrate according to claim 6, wherein in (b) the pore size is between 30 and 200 μm.

8. A synthetic cartilage patch for the repair of a cartilage defect in a mammal in vivo comprising synthetic cartilage formed on a substrate of claim 1.

9. The synthetic cartilage patch according to claim 8 wherein the cartilage is characterized by an about 1.5 fold higher cellularity and an about 1.5 fold higher proteoglycan content as compared to cartilage tissue formed on a substrate with interconnected pores having an average pore size greater than 40 μm.

10. The synthetic cartilage patch according to claim 8, wherein the synthetic cartilage is synthetic articular cartilage.

11. A method for preparing in vitro a synthetic cartilage patch for the repair of a cartilage defect in a mammal comprising:
(a) preparing a substrate comprising forming from a material capable of forming pores with a selected pore size, a porous construct with interconnected pores having an average pore size less than 70 μm; and
(b) culturing denuded chondrogenic cells on the substrate under conditions sufficient to permit the cells to form a three-dimensional multi cell-layered patch of synthetic cartilage.

12. The method according to claim 11, wherein in (a) the material is a powder selected from the group consisting of calcium phosphates, titanium or titanium alloy (Ti6Al4V), hydroxyapatite, and calcium carbonate.

13. The method according to claim 12, wherein the powder has a particle size less than 100 μm.

14. The method according to claim 11, wherein the chondrogenic cells are isolated from mammalian articular cartilage and the synthetic cartilage is synthetic articular cartilage.

15. The method according to claim 11, wherein the porous construct in (a) is formed on or with, or placed on a deeper component comprising a porous construct with a pore size selected to permit bone ingrowth in the substrate.

16. The method according to claim 15 wherein the pore size of the deeper component is between 30 and 200 μm.

17. A synthetic cartilage patch prepared by the method of claim 11, wherein the synthetic cartilage is characterized by an about 1.5 fold higher cellularity, and an about 1.5 fold higher proteoglycan content as compared to synthetic cartilage formed on a substrate with interconnected pores having an average pore size greater than about 40 μm.

18. A method for effecting the repair of a cartilage defect at a pre-determined site in a mammal comprising:
(a) surgically implanting at the pre-determined site a synthetic cartilage patch of claim 17; and
(b) permitting the synthetic cartilage of the patch to integrate into the pre-determined site.

19. The method according to claim 18, wherein the defect is a partial-thickness or full-thickness defect of articular cartilage.

20. A system for testing a substance that affects cartilage tissue comprising:
(a) culturing denuded chondrogenic cells on a substrate of claim 1 under conditions to permit the cells to form a three-dimensional multi cell-layered patch of synthetic cartilage in the presence of a substance which is suspected of affecting formation or maintenance of cartilage, and
(b) determining the biochemical composition and/or physiological organization of the synthetic cartilage generated in the culture with the biochemical composition and/or physiological organization of the synthetic cartilage in the absence of the substance.

21. A method of using a synthetic cartilage patch of claim 17 to test pharmaceutical preparations for efficacy in the treatment of diseases of the joint.
